(19) United States
(12) Patent Application Publication
(10) Pub. No.: US 2006/0178750 A1
(43) Pub. Date: Aug. 10, 2006

(54) Nature hipbone device

(76) Inventor: Poonung Chieng, Shanghai (CN)

Correspondence Address: BACON & THOMAS, PLLC, 625 SLATERS LANE, FOURTH FLOOR, ALEXANDRIA, VA, 22314, US

(21) Appl. No.: 10/549,095

(22) PCT Filed: Mar. 15, 2004
(86) PCT No.: PCT/CN04/00204

§ 371 Date: Sep. 13, 2005

(30) Foreign Application Priority Data
Mar. 13, 2003 (CN) 03115791.2

(51) Int. Cl. A61F 2/34

(52) U.S. Cl. 623/22.27; 623/22.28

(57) ABSTRACT

The invention relates a nature hipbone device which includes a cotyle cup, an orientation pivot which localizes the cotyle cup in its place, is fixed on the close end of the cotyle cup; at least one link member which is hinged onto the opening edge of the end of the cotyle cup, at least one lock member which sleeve on the edge of an end of the link member apart from the cotyle cup may be located on secure place by the lock member.

### BACKGROUND OF THE INVENTION

1. Field of the Invention

The present invention relates to a nature hipbone device, more particularly to a nature hipbone device installed onto an artificial femoral head, and its accessories are fixed to secure a hip joint so that the device will not shift or fall off as the hip joint is moving.

2. Description of the Related Art

For the treatment of an injured femoral neck, the damaged part is generally cut away or sawn even and covered by a sheet steel, and then two nails are installed into the femur for fixing the sheet steel. However, the design of such a traditional structure has the following shortcomings: (1) The sheet steel cannot be secured; and (2) The newly grown blood vessels and bone crusts cannot cover the sheet steel effectively, so that the sheet steel cannot be integrated completely with the femur in order to protect the femur. The force exerted on the sheet steel at the damaged part is very limited, and thus the sheet steel will be separated from the femur easily. Further, the sheet steel cannot move according to the movements of the hip joint, which makes the application useless and the patient painful. The abovementioned shortcomings have been major issues for medical people and patients.

To improve the foregoing shortcomings, a hip joint structure as shown in FIG. 1 has been introduced. The device includes an artificial joint stem 100 which is substantially curved and corresponding to the shape of the femur. The artificial joint stem 100 is tapered from one end to another end, and the end of the artificial joint stem 100 having a larger cross-sectional area is connected to an artificial femur adjustable joint 110. A metal cotyle cup 120 is embedded into the artificial femur adjustable joint 110, and a lining made of a macromolecular material (polyethylene) is disposed between the cotyle cup 120 and the artificial femur adjustable joint 110.

When the device is in use, the damaged femur is cut away, and the femoral neck is processed properly, and then the artificial joint stem 100 is inserted into the femur (not shown in the figure), such that the artificial joint stem 100 is penetrated deeply into the femur cavity and integrated with the femur. In the meantime, the metal cotyle cup 120 is fixed into the hip joint by metal nails 121, and finally the artificial femur adjustable joint 110 is sheathed into the cotyle cup 120. However, the application of this device has the following drawbacks:

1. Since both of the artificial femur adjustable joint 110 and the cotyle cup 120 are made of metal, therefore it is necessary to have the polyethylene lining 130 installed between the artificial femur adjustable joint 110 and the cotyle cup 120 to serve as a cushion to reduce frictions. After a long time of use, the polyethylene lining 130 will be worn into debris, which will cause a soluble bone and a loosened cotyle cup.

2. Since the metal cotyle cup 120 is secured onto the hip joint by metal nails 121 and the nails are located within a specific area, therefore the cotyle cup 120 will be loosened or fallen off easily if it is pressed by the hip joint.

### SUMMARY OF THE INVENTION

Therefore, it is the primary objective of the present invention to provide a nature hipbone device which is installed onto an artificial femoral head, and its accessories are fixed to secure the hip joint so that the device will not shift or fall off as the joint moves.

The nature hipbone device comprises a cotyle cup, and the internal wall of the cotyle cup is a very precise and smooth wall surface for directly receiving an artificial femoral head and minimizing the friction. The cotyle cup includes an orientation pivot and at least one pair of symmetrical link member disposed at the edge of an open end of the cotyle cup, a lock member disposed at a free end of each of the plurality of link member. When the nature hipbone device is in use, the femoral head is cut away and the femoral neck is kept, and then an artificial joint stem pivotally coupled to the artificial femur adjustable joint is inserted into the femur cavity and fixed at a predetermined position by the orientation pivots and locked into the hipbone by the lock member. As a result, the cotyle cup can be fixed into its position by means of the orientation pivots and the lock member at different positions and the cotyle cup can be integrally secured into the hip joint. The artificial femur adjustable joint is sheathed into the cotyle cup, so that the cotyle cup can move according to the movements of the hip joint, and the invention can prevent the cotyle cup from being slid or loosened.

Further, the cotyle cup includes a cup member corresponding to the shape of the cotyle cup, and the cup member includes a plurality of ribs around the cup member. When the cotyle cup is used, the femoral head is cut away and the femoral neck is kept, and then an artificial joint stem pivotally coupled to the artificial femur adjustable joint is inserted into the femur cavity and the cotyle cup is fixed at a predetermined position by the orientation pivots, and passed through the ribs embedded in the hipbone, so that the cotyle cup can be fixed into its position and secured to the hip joint. The artificial femur adjustable joint is sheathed into the cotyle cup, such that when the cotyle cup moves according to the movements of the femoral joint, the cotyle cup will not be slid or loosened by the pressing of the joint.

Further, when the cotyle cup is used, the femoral head is cut away and the femoral neck is kept, and then an artificial joint stem pivotally coupled to the artificial femur adjustable joint is inserted into the femur cavity and the cotyle cup is fixed at a predetermined position by the orientation pivots, and the through holes disposed on the cup member allow blood vessels and muscles to be penetrated deeply therein and integrated with the cotyle cup. Therefore, the cotyle cup can be fixed into its position. The artificial femur adjustable joint is sheathed into the cotyle cup, so that when the cotyle cup moves according to the movements of the femoral joint, the cotyle cup will not be slid or loosened by the pressing of the joint.

Further, the cotyle cup and the artificial femur adjustable joint can be engaged with each other, so that the cotyle cup will not be separated from the artificial femur adjustable joint.

### BRIEF DESCRIPTION OF THE DRAWINGS

FIG. 1 is a schematic view of a prior art nature hipbone device;

FIG. 2 is an exploded view of a nature hipbone device according to a preferred embodiment of the present invention;

FIG. 3 is a perspective view of a nature hipbone device according to a preferred embodiment of the present invention;

FIG. 4 is a cross-sectional view of a nature hipbone device according to another preferred embodiment of the present invention;

FIG. 5 is a cross-sectional view of a nature hipbone device according to a further preferred embodiment of the present invention; and

FIG. 6 is a perspective view of a cup member according to a further preferred embodiment of the present invention.

### DETAILED DESCRIPTION OF THE PREFERRED EMBODIMENTS

The above and other objects, features and advantages of the present invention will become apparent from the following detailed description taken with the accompanying drawing.

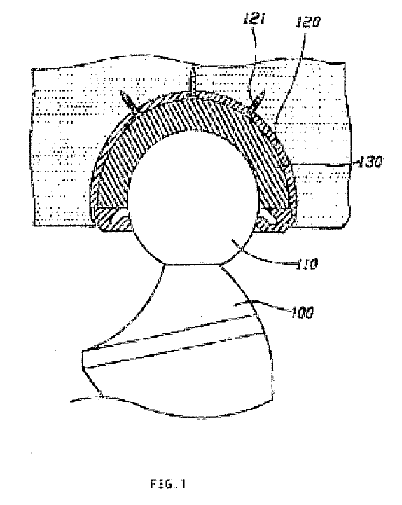

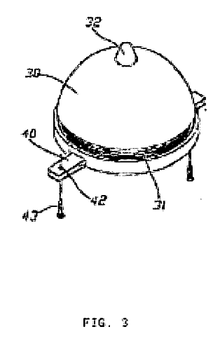

Referring to FIGS. 1 to 3 for the preferred embodiment of the present invention, a nature hipbone device of the invention comprises an artificial joint stem 100 being substantially in a curved shape corresponding to the shape of the femur. The artificial joint stem 100 is tapered from one end to the other end, and the end of the artificial joint stem 100 having a larger cross-sectional area is sheathed into a cotyle cup 30 made of a metal. The cotyle cup 30 adopts a nano technology or other method to make a very smooth surface of its walls, so that when the artificial femur adjustable joint 110 and the cotyle cup 30 are coupled with each other, almost no friction exists between the artificial femur adjustable joint 110 and the cotyle cup 30.

Further, the shape of the cotyle cup 30 corresponds to the shape of the artificial femur adjustable joint 110, and the cotyle cup 30 includes an orientation pivot disposed at the edge of an open end of the cotyle cup 30. The cotyle cup 30 further includes a thread 31 disposed proximate to the edge surface of an open end of the cotyle cup 30 for being screwed with a cup member 50. The cup member 50 includes a plurality of through holes 52 thereon, so that blood vessels and muscles can be penetrated therein. The cup member 50 includes a hollow hole 51 corresponding to the orientation pivot 32, and the cotyle cup 30 includes at least one pair of symmetrical concave openings 33 disposed at the edge of the opening of the cotyle cup 30. The concave openings 33 separately include a link member 40 embedded in the concave opening 33, and the link member 40 is an n-shaped frame in this preferred embodiment. The plurality of link member 40 include a pivot 41 embedded at a closed end of the link member 40, and both sides of the pivot 41 press against the internal side of the cotyle cup 30 to fix the link member 40 onto the cotyle cup 30 and couple with the cotyle cup 30. An open hole 42 is disposed at an open end of the link member 40 for passing the lock member 43, and the lock member in this preferred embodiment are screws.

Referring to FIGS. 1 to 3 for the use of the nature hipbone device, the damaged femoral head is cut away and the femoral neck is processed properly, and then the artificial joint stem 100 is inserted into the femur cavity (not shown in the figure), and penetrated deeply into the femur cavity to integrate with the femur. In the meantime, the metal cotyle cup 30 is penetrated into the hip joint by the orientation pivots and secured into a fixed position, and then locked by the lock member 43, and thus the cotyle cup 30 can be fixed onto the hip joint. The artificial femur adjustable joint 110 is sheathed into the cotyle cup 30, so that the cotyle cup 30 can move according to the movement of the hip joint, and the cotyle cup 30 will not be slid or loosened easily by the pressing of the hip joint.

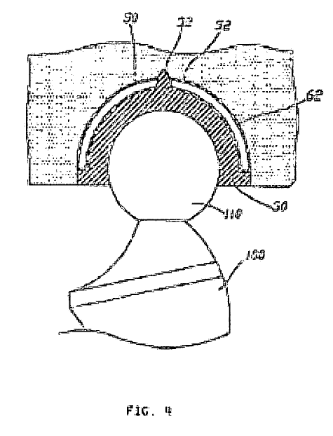

Referring to FIG. 4 for another preferred embodiment of the present invention, the device includes an artificial joint stem 100 substantially in a curved shape and is tapered from one end to the other end. An end having a larger cross-sectional area is coupled with an artificial femur adjustable joint 110 and the artificial femur adjustable joint 110 is engaged with a cotyle cup 30 made of metal. The cotyle cup 30 adopts a nano technology or other methods to produce a very smooth wall surface for the internal wall of the cotyle cup 30, such that when the artificial femur adjustable joint 110 and the cotyle cup 30 are engaged, almost no friction exists between the artificial femur adjustable joint 10 and the cotyle cup 30.

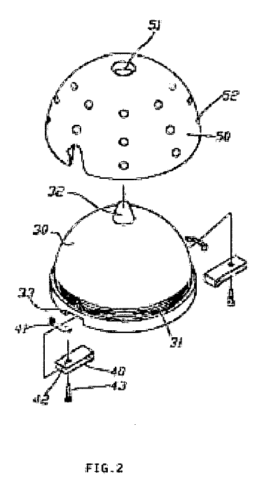

Further, the cotyle cup 30 includes an orientation pivot 32 disposed at a closed end of the cotyle cup 30 and a cup member 50 (as shown in FIG. 2) having a corresponding shape with the cotyle cup 30 and screwed onto the cotyle cup 30. The cup member 50 includes a concave opening 51 corresponding to a protruded fixing pillar 32 and a through hole 52 disposed around the periphery of the cup member 50.

Referring to FIG. 4 for the use of the nature hipbone device, the damaged femoral head is cut away and the femoral neck is processed properly, and then the artificial joint stem 100 is inserted into the femur cavity (not shown in the figure), such that the artificial joint stem 100 can be penetrated into the femur cavity to integrate the artificial joint stem 100 with the femur. In the meantime, the metal cotyle cup 30 is engaged with the hip joint by the orientation pivots 32 and secured at a fixed position and passed through the through hole 52, so that blood vessels and muscles can penetrate into the cup member 50 and the cotyle cup 30 can be fixed onto the hip joint to sheath the artificial femur adjustable joint 20 into the cotyle cup 30. The cotyle cup 30 can move according to the movements of the hip joint, and the cotyle cup 30 will not be slid or loosened easily by the pressing of the hip joint.

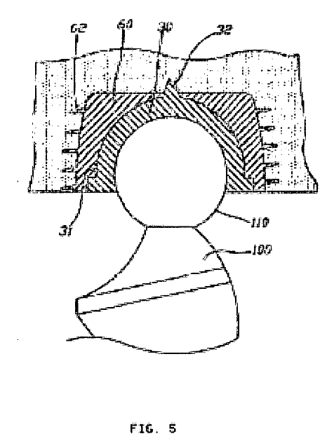

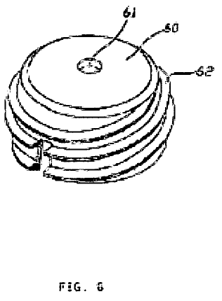

Referring to FIGS. 5 and 6 for the further preferred embodiment of the present invention, the device includes an artificial joint stem 100 that has a curved shape corresponding to the shape of a femur, and the artificial joint stem 100 is tapered from one end to the other end. An end of the artificial joint stem 100 having a larger cross-sectional area is coupled to an artificial femur adjustable joint 20. The artificial femur adjustable joint 20 includes a metal cotyle cup 30. The cotyle cup 30 adopts a nano technology or other methods to produce a very smooth surface for the wall of the cotyle cup 30, such that when the artificial femur adjustable joint 20 and the cotyle cup 30 are engaged, almost no friction exists between the artificial femur adjustable joint 20 and the cotyle cup 30.

Further, the cotyle cup 30 includes an orientation pivot 32 and a thread 31. The thread 31 is screwed onto a corresponding cup member 60 as shown in FIG. 5. The cup member 60 includes a hollow hole 61 corresponding to the protruded fixing pillar 32, and a rib 62 disposed around the cup member 60.

Referring to FIGS. 5 and 6 for the use of the nature hipbone device, the damaged femoral head is cut away and the femoral neck is processed properly, and then the artificial joint stem 100 is inserted into the femur cavity (not shown in the figure), such that the artificial joint stem 100 is penetrated into the femur cavity to integrate the artificial joint stem 100 with the femur. In the meantime, the metal cotyle cup 30 is penetrated into the hip joint through the orientation pivots 32 and secured at a fixed position. With the rib 62 embedded into the hip joint, the cotyle cup 30 can be secured onto the hip joint, and then the artificial femur adjustable joint 110 is sheathed into the cotyle cup 30. Therefore, the cotyle cup 30 can move according to the movements of the hip joint, and the cotyle cup 30 will not be slid or loosened easily by the pressing of the hip joint.

What is claimed is:

1. A nature hipbone device, comprising:
a cotyle cup, having an orientation pivot disposed at a closed end of said cotyle cup for fixing said cotyle cup;
more than one link member, pivotally coupled to the edge of an open end of said cotyle cup; and
more than one lock member, passing through an end away from said cotyle cup for locking said cotyle cup into a fixed position.

2. The nature hipbone device of claim 1, wherein said cotyle cup includes a precise smooth surface disposed at the internal wall of said cotyle cup.

3. The nature hipbone device of claim 1, wherein said lock member is a screw.

4. The nature hipbone device of claim 1, wherein said cotyle cup includes at least one pair of symmetric concave openings disposed on the opening edge of the end of said cotyle cup and a pivot is disposed in said concave opening for fixing said link member with said cotyle cup.

5. The nature hipbone device of claim 1, wherein said cotyle cup includes a thread thereon for screwing to a cup member having a shape corresponding to said cotyle cup, and said cup member includes a plurality of through holes thereon.

6. The nature hipbone device of claim 1, wherein said cotyle cup includes a thread thereon for screwing to a cup member having a shape corresponding to said cotyle cup, and said cup member includes a plurality of ribs disposed around said cup member.

7. The nature hipbone device of claim 5, wherein said cup member includes a hollow hole corresponding to said orientation pivot.

8. The nature hipbone device of claim 6, wherein said cup member includes a hollow hole corresponding to said orientation pivot.

9. A nature hipbone device, comprising:
a cotyle cup, having an orientation pivot disposed at a closed end of said cotyle cup for fixing said cotyle cup; and
a cup member, screwed onto said cotyle cup and having a rib disposed around said cup member, such that said cotyle cup is capable of coupling an attached object.

10. The nature hipbone device of claim 8, wherein said cotyle cup includes a precise smooth surface disposed at the internal wall of said cotyle cup.

11. The nature hipbone device of claim 8, wherein said cup member includes a hollow hole corresponding to a protruded fixing pillar.

12. The nature hipbone device of claim 8, wherein said cup member includes a through hole.

13. The nature hipbone device of claim 8, wherein said cotyle cup includes a thread.

14. The nature hipbone device of claim 8, wherein said cotyle cup includes at least one pair of symmetric concave openings disposed on the opening edge of the end of said cotyle cup and a pivot is disposed in said concave opening for fixing said link member with said cotyle cup.

15. A nature hipbone device, comprising:
a cotyle cup, having an orientation pivot disposed at a closed end of said cotyle cup for fixing said cotyle cup; and
a cup member, screwed onto said cotyle cup and having a plurality of through holes disposed on said cup member for allowing blood vessels and muscles to be penetrated therein and covering said cotyle cup, such that said cotyle cup is capable of coupling an attached object.

16. The nature hipbone device of claim 14, wherein said cotyle cup includes a precise smooth surface disposed at the internal wall of said cotyle cup.

17. The nature hipbone device of claim 14, wherein said cup member includes a hollow hole corresponding to a protruded fixing pillar.

18. The nature hipbone device of claim 14, wherein said cup member includes a rib.

19. The nature hipbone device of claim 14, wherein said cotyle cup includes a thread.

20. The nature hipbone device of claim 14, wherein said cotyle cup includes at least one pair of symmetric concave openings disposed on the opening edge of the end of said cotyle cup and a pivot is disposed in said concave opening for fixing said link member with said cotyle cup.
